(19) United States
(12) Patent Application Publication
(10) Pub. No.: US 2024/0285511 A1
(43) Pub. Date: Aug. 29, 2024

(54) CANNABINOID-CONTAINING FORMULA FOR SKIN CARE

(71) Applicant: LONDON PHARMACEUTICALS AND RESEARCH CORPORATION, London (CA)

(72) Inventors: Abdelrahman S. Mayhoub, Giza (EG); Mahmoud Mohamed Abdrabo Moustafa, London, Ontario (CA)

(21) Appl. No.: 18/572,696

(22) PCT Filed: Jun. 23, 2022
(86) PCT No.: PCT/CA2022/051012

§ 371 Date: Dec. 20, 2023

Related U.S. Application Data
(60) Provisional application No. 63/213,812, filed on Jun. 23, 2021.

(51) Int. Cl. A61K 8/9789; A61K 8/34; A61K 8/66; A61K 8/73; A61K 8/92; A61Q 19/00; A61Q 19/08

(52) U.S. Cl. CPC A61K 8/9789; A61K 8/347; A61K 8/66; A61K 8/735; A61K 8/922; A61Q 19/00; A61Q 19/08

(57) ABSTRACT

The present invention relates to the formulation and the use of a cannabinoid, hyaluronate and certain botanical extracts or phytochemical materials to improve the health of skin. The formulations address the incompatibility between oil-soluble cannabinoids and aqueous herbal extracts, or water-soluble phytochemical components. Preferably, the botanical extract is selected from Opuntia ficus-indica, Carica papaya, Vasconcellea pubescens, Salvadora persica, Nigella sativa, Glycyrrhiza glabra, Camellia sinensis, and Aloe vera.

### FIELD OF THE INVENTION

The present invention relates to the field of skin care and, in particular, to antiaging, antiwrinkle, and skin whitening.

### BACKGROUND

Hyaluronic acid is a polymer of glucuronic acid and N-acetylglucosamine disaccharide. It is a powerful humectant (1 g hyaluronic acid can hold up to 6 L of water) and is widely recognized to play a key role in skin wellness. It holds water and maintains the extracellular space and is important for skin hydration and elasticity. It is important in maintaining the look and feel of softer, smoother, and radiantly hydrated skin.

Many components of hemps oil, for instance cannabidiol (CBD), are beneficial in reversing the process of skin aging. CBD, and other cannabinoids, exert their antiaging possess via different mechanisms, for example: 1) they possess the ability to counteract free-radical damage and reduce the look of inflammation; 2) they can slow down collagen loss, and help the skin retain its natural tautness and firmness; or 3) they can act to modulate the endocannabinoid system directly or indirectly in the skin.

Many herbal extracts and phytochemical components are also known with their direct or indirect benefits on skin care. This list includes: Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.) Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evernia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichrysum (Helichrysum italicum); Grapefruit, White (Citrus x paradisi); Grapefruit, Pink (Citrus paradisi); Ginger (Zingiber officinalis); Geranium (Pelargonium graveolens); Geranium, Bourbon (Pelargonium graveolens,'Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, and Eucalyptus Citriodora.

Combinations of CBD, other related cannabinoid(s) or hemp oil, and herbal extracts, for the purposes of skin antiaging, have been reported in many patents, for example: US20150245991A1, US20160235661A1, CN108685729A, WO2019140357A1, CN110613642A, CN110559230A, WO2019195943A1, CN110215406A, CN109998941A, CN110051568A, CN109939021A, CN110339123A, CN111956535A, CN110664636A, CN110664637A, CN110787113A, CN111228182A, CN112353702A, CN112691068A, CN112451399A, CN112641655A, CN112516120A, WO2017218845A1, WO2019198018A1, CN111956543A, CN112386541A, and CN111956543A.

Although promising, these combinations are encumbered by the fact that the extracts from different plants or even different parts from the same plant (e.g., fruit versus stem) may exhibit substantially different physical, chemical, and biological properties. In particular, the pharmacologically active extract from hemp and/or cannabis is oil-soluble, while the majority of antiaging herbal extracts are water-soluble and, as a result, compounding them in a stable formula remains a major formulation challenge. Furthermore, many of these combinations do not use the part of the plant that provides the maximum synergistic antiaging effect when combined with cannabinoids.

To minimize the limitations in the prior art, there exists a demand for new synergistic and effective antiaging formulas with optimized physicochemical, pharmacological and cosmetic properties.

### SUMMARY OF THE INVENTION

The present invention provides compositions and methods for treating conditions and/or symptoms, or alleviating or delaying symptoms of certain dermatological conditions, including symptoms of aging, wrinkles, dryness, and skin darkness, comprising administering to a subject in need thereof a therapeutically effective amount of a skin care formulation comprising:
a. at least one cannabinoid, preferably CBD or hemp oil;
b. at least one herbal extract, preferably fruit extract of Opuntia ficus-indica;
c. at least 1×10^{-4% by weight of a hyaluronate derivative, preferably the sodium salt;}
d. at least 5×10^{-3% by weight of a pharmaceutically acceptable skin penetration enhancer;}
e. at least 2×10⁻³% by weight of a pharmaceutically acceptable oleaginous base;
f. at least 5×10^{-3% by weight of a pharmaceutically acceptable emollient;}
g. Optionally, at least 0.1% by weight of additional herbal extract(s) or phytochemical component(s), preferably Carica papaya, Mountain papaya, Salvadora persica, Nigella sativa, Glycyrrhiza glabra, Camellia sinensis, and Aloe vera;
h. Optionally, at least 1×10^{-4% by weight of a pharmaceutically acceptable exfoliating agent;}
i. Optionally, at least 1×10^{-4% by weight of a pharmaceutically acceptable UV blocking agent;}
j. Optionally, at least 2×10⁻⁵% by weight of a pharmaceutically acceptable antioxidant;
k. Optionally, at least 7.5×10^{-4% by weight of a pharmaceutically acceptable emulsifying agent;}
l. Optionally, at least 1×10⁻³% by weight of a pharmaceutically acceptable co-emulsifying agent;
m. Optionally, at least 5×10⁻³% by weight of a pharmaceutically acceptable stabilizer;
n. Optionally, at least 5×10⁻³% by weight of a pharmaceutically acceptable solubilizing agent;
o. Optionally, at least 1×10⁻³% by weight of a pharmaceutically acceptable viscosity modifying agent;
p. Optionally, at least 1×10⁻⁴% by weight of a pharmaceutically acceptable preservative;
q. Optionally, at least 1×10⁻⁴% by weight of a pharmaceutically acceptable natural preservative material;
r. Optionally, at least 1×10⁻⁴% by weight of a pharmaceutically acceptable natural perfuming agent;
s. Optionally, at least 1×10⁻⁴% by weight of a pharmaceutically acceptable flavoring agent; and
t. Optionally, at least 1×10^{-4% by weight of one or more additional pharmaceutically acceptable coloring agents, pH modifiers or buffers.}

In the present invention, combining the inflammatory cytokines suppression effect of CBD with the powerful antioxidant properties of Opuntia fruit extract in one product offers a synergistic combination, useful for several skin care applications.

### DESCRIPTION OF THE INVENTION

The present invention discloses a topical preparation that comprises one or more cannabinoid component(s), herbal extract(s), or phytochemical components and hyaluronate derivative. The present invention also discloses methods of treating, alleviating, or delaying certain dermatological conditions or symptoms by administering to a subject in need, a therapeutically effective amount of a compound comprising one or more cannabinoid component(s), herbal extract(s), or phytochemical components and hyaluronate derivative.

The components of compounds of the present invention cooperate with one another to provide synergistic effects or to improve the formulation characteristics, delivery, availability, or other characteristics of one or more other components of the compound.

The fruit extract of Opuntia ficus-indica is characterized by the presence of a phenolic glycoside called betanin, as a main component (Kim, Seung Hyun, et al. Journal of medicinal food 15.11 (2012): 968-973), which possesses strong free radical scavenging activity (Esatbeyoglu, Tuba, et al. Food and Chemical Toxicology 73 (2014): 119-126). The other characteristic component of Opuntia ficus-indica fruit extract is indicaxanthin, which is a more powerful antioxidant than vitamin E (Butera, Daniela, et al. Journal of agricultural and food chemistry 50.23 (2002): 6895-6901). As a result, the Opuntia fruit extract has significant therapeutic benefits and can be used in various applications for skin care.

Additionally, several cannabinoids are reported to modulate the cannabinoid receptors in keratinocytes and sebocytes. The mechanism involves suppression of the synthesis and the secretion of several inflammatory cytokines such as IL-2 and IL-23 (Chiurchiù et al. (2016) J Immunol. 197: 3545-3553). Hence, cannabinoids can decrease skin inflammation and skin discomfort, in addition to other dermatological reactions such as pruritus, and are similarly useful as antiaging compounds.

In another embodiment, the herbal extract component is the extract of any edible Opuntia species, which may be used alone or in combination with any other phytochemical compound(s), other extract(s), nutritional supplements, vitamins (e.g., Vit A, B6, Vit C), fatty acids (e.g., oleic acid), amino acids (e.g., arginine), peptides, carbohydrates, sugars and/or therapeutic agent(s).

In some embodiments, the herbal extract component is a papaya species extract (e.g., Carica papaya, Vasconcellea pubescens (Mountain papaya)), or proteolytic enzymes, such as papain and bromelain.

Papain, isolated from Papaya species, has a proteolytic effect with the ability to remove dead skin. It facilitates the therapeutic action of other components by allowing them to be in contact with the living part of the skin.

In some embodiments, hyaluronic acid, or its salts, preferably the sodium salt, or analogues or derivatives are used as humectants and skin moisturizing agents. In additional to, or in place of, hyaluronic acid salts, analogues, or derivatives, any hyaluronate salts, esters, or any of its precursors, lysates or partial lysates, or combination thereof may be used.

Skin aging is associated with loss of skin moisture and hence appearance of skin wrinkles. The key molecule involved naturally in skin moisture is hyaluronic acid. Therefore, the compounds of the present invention include hyaluronic acid, with its powerful hydration property, in addition to skin penetrating agent(s) with the aim of replenishing the hydration capacity of aged skin.

In some embodiments, the cannabinoid component is one or more cannabinoids selected from the group consisting of delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof. Preferably, the cannabinoid is CBD or hemp oil.

In some embodiments, compounds of the present invention can comprise one or more additional herbal extracts, isolated active component(s), or mixture or derivatives thereof, independently selected from a group that includes: Myrrh, Myrrh resin, Myrrh volatile oil, or myrrhin, Salvadora persica, Nigella sativa, Glycyrrhiza glabra, Camellia sinensis, Aloe vera, terpene (e.g. β-Caryophyllene), collagen, Albizia Julibrissin, Squalane, Prunus Amygdalus Dulcis, Tephrosia Purpurea, probiotic (e.g. Bifida Ferment Lysate), Adansonia Digitata, Yeast, Extrait De Levure, Cola Acuminata, Anthemis Nobilis, Curcuma Longa (Turmeric), Dipsacus Sylvestris, Silybum Marianum, Chenopodium Quinoa Seed, Avena Sativa (Oat) Kernel, Theobroma Cacao, Leontopodium Alpinum, Musa Sapientum, Salicornia Herbacea, Actinidia Chinensis, Kalanchoe Pinnata, Myrothamnus Flabellifolia, Lycium Barbarum, Orthosiphon Stamineus, Hedychium Coronarium, Mangifera Indica, Jania Rubens, Engelhardtia Chrysolepis, Callicarpa Japonica, Retinoids, argan, tiger grass, shea butter, adapalene, hydroquinone, limnanthes alba (meadowfoam), coconut, withania somnifera (ashwagandha), Helianthus annuus (sunflower), Helianthus annuus, lentinus edodes (shiitake) extract), Borago officinalis, tocopheryl acetate (vitamin E), Helichrysum stoechas, Melia azadirachta, Melia azadirachta, amino esters, Mimosa tenuiflora, Coccinia indica, Solanum melongena (eggplant), Aloe barbadensis, Ocimum sanctum, Ocimum basilicum, Curcuma longa (turmeric), Corallina ocinalis, Marula, Argan, Lavender Oil, Sunflower, Simmondsia chinensis (jojoba), Prunus armeniaca (apricot) kernel, Borago officinalis (borage), Punica granatum (pomegranate), Cucumis sativus (cucumber), Vaccinium macrocarpon (cranberry), Brassica oleracea italica (broccoli), Persea gratissima (avocado), Coffea arabica (green coffee), Calendula officinalis (Calendula), Centella asiatica (gotu kola), Equisetum arvense (horsetail), Urtica dioica (nettle), Hippophae rhamnoides (sea buckthorn) fruit, Rosmarinus officinalis (rosemary) extract, Citrus aurantium (bergamot), Citrus limon (lemon), Pelargonium graveolens (geranium), Boswellia carteri (frankincense), Jasminum grandiflorum (jasmine), Citrus aurantium (neroli), Daucus carota (carrot seed), sea buckthorn, Calendula, sandalwood, and tea rose.

In some embodiments, compositions of the present invention can comprise one or more stilbene antioxidant derivatives, such as piceatannol and resveratrol.

In some embodiments, compositions of the present invention can comprise one or more additional compounds or derivatives thereof, including exfoliating agents, such as salicylic acid, glycolic acid, malic acid, citric acid, and the like, their salts, hydrates, or combinations thereof.

In some embodiment, compositions of the present invention can comprise one or more additional compounds or derivatives thereof, including natural, synthetic, or semisynthetic UV blocking agents, such as curcumin derivatives, coconut oil, titanium dioxide, zinc oxide, and butyl methoxy dibenzoylmethane, para-aminobenzoic acid and its derivatives, cinnamates such as octyl methoxy cinnamate, salicylates, camphor derivatives, avobenzone, triazones, benzophenones, and the like or combinations thereof. Preferably, when using titanium dioxide or zinc oxide, they are highly micronized to the sub-micro scale.

In addition to the above active components, the compounds may optionally contain additional excipients or inactive materials. The role of these additional material(s) varies from stabilizing the composition, facilitate manufacturing process(es), prolonging the shelf-life, augmenting the effect of active component(s), facilitating the active component(s) to reach their site of action, enhancing the appearance and subject/patient acceptability, or providing other additional positive aspect(s) to the formula.

In some embodiments, the composition is a cosmetic or pharmaceutical composition, further comprising a pharmaceutically acceptable excipient.

In some embodiments, compositions of the present invention can comprise one or more additional compounds or derivatives thereof, including but not limited to skin penetration enhancer(s), such as dimethyl sulfoxide (DMSO), lanolin, wool fat, polyethylene glycol with any grade (Macrogols) or blend, caprylic acid triglyceride (Tricaprylin), and myristate esters, including methyl, ethyl, propyl, isopropyl, butyl, isobutyl, tert-butyl and the like; preferably isopropyl myristate.

In some embodiments, the skin penetrating enhancer(s) is/are used in an amount of 1%, 2%, 5%, 7%, 10%, 12%, 15%, or up to 35% by weight.

In some embodiments, compositions of the present invention comprise ingredients including, an oleaginous base or a cream base. This includes any suitable oily or waxy material, such as liquid paraffin, mineral oil, white mineral oil, any vegetable oil such as sunflower oil, peanut oil, almond oil, olive oil, (Ω-3 fatty acid(s) such as eicosapentaenoic acid, α-linolenic acid, fish oil, triglycerides and in particular medium chain triglycerides, cetostearyl ether, macrogol lauryl ether, macrogol oleyl ether, macrogol stearyl ether, lauromacrogol, hydrogenated polyoxyl castor oil, fatty acid esters such as ethyl stearate, mixture or blend thereof.

In some embodiments, the oleaginous base is used in an amount of 5%, 7%, 10%, 12%, 15%, 20%, 25%, or up to 99% by weight.

In some embodiments, compositions of the present invention can comprise one or more additional compounds or derivatives thereof, including additional antioxidant(s), such as vitamin A, vitamin E, vitamin C, ascorbyl palmitate, ascorbate salt(s), any hydroxytoluene derivatives, preferably butylated hydroxytoluene (BHT), any hydroxyanisole, preferably butylated hydroxyanisole (BHA), pineapple extract, citric acid monohydrate, edetic acid, erythorbic acid, fumaric acid, malic acid, methionine, monothioglycerol, pentetic acid, phosphoric acid, metabisulphite salt(s), propionic acid, gallate ester(s) such as propyl or ethyl, sodium formaldehyde sulfoxylate, sodium pyrosulfite, sulfite salt(s), thiosulphate salt(s), sulfur dioxide, tartaric acid, thymol, vitamin E polyethylene glycol succinate, glycyrrhizic acid, and the like, mixture or blend thereof.

In some embodiments, the antioxidant or mixture/blend of antioxidants is used in an amount of 0.01%, 0.02%, 0.5%, 0.7%, 1%, 2%, 5%, or up to 35% by weight.

In some embodiments, compositions of the present invention comprise ingredients including, emulsifying agents including anionic, cationic or non-ionic surfactants, such as tween 20, tween 40, tween 60, tween 80, span 20, span 40, span 60, span 80, acacia, agar, bentonite, calcium alginate, carbomer, carboxymethylcellulose, carrageenan, cetostearyl alcohol, cetyl alcohol, cholesterol, diethanolamine, docusate salts, glyceryl monooleate, glyceryl monostearate, glyceryl palmitostearate, glyceryl monostearate, hectorite, hydroxypropyl cellulose, hydroxypropyl starch, lanolin, hydrous lanolin, lanolin alcohols, lauric acid, lecithin, linoleic acid, magnesium oxide, methylcellulose, Amerchol L-101, monoethanolamine, myristic acid, myristyl alcohol, octyldodecanol, oleic acid, oleyl alcohol, pectin, phenylmercuric salts, phospholipids, poloxamer, polycarbophil, polyoxyethylene alkyl ethers, hydrogenated polyoxyl castor oil, polysorbate 20, polysorbate 40, polysorbate 60, polysorbate 90, polyoxyethylene stearate(s), polyoxylglyceride(s), caprylocaproyl macrogolglycerides, lauroyl macrogolglycerides, linoleoyl macrogolglycerides, oleoyl macrogolglycerides, stearoyl macrogolglycerides, potassium alginate, propylene glycol alginate, safflower glyceride(s), saponite(s), borax, sodium citrate, sodium lactate, sodium lauryl sulphate, sodium phosphate, sorbitan esters, sorbitan laurate, sorbitan oleate, sorbitan palmitate, sorbitan stearate, sorbitan trioleate, sorbitan sesquioleate, stearic acid, stearate salt(s), stearate esters, triethanolamine, vitamin E polyethylene glycol succinate, emulsifying wax, cetostearyl alcohol (type A), cetostearyl alcohol (Type B), cetomacrogol emulsifying wax, xanthan gum, preferably cetomacrogol 1000 (ceteareth-20) and carbopol 940.

In some embodiments, the emulsifying agent(s) is/are used in an amount of 0.1%, 2%, 5%, 7%, 10%, 12%, 15%, or up to 35% by weight.

In some embodiments, compositions of the present invention comprise ingredients including, co-emulsifying agents including anionic, cationic or non-ionic surfactants, such as tween 20, tween 40, tween 60, tween 80, span 20, span 40, span 60, span 80, acacia, agar, bentonite, calcium alginate, carbomer, carboxymethylcellulose, carrageenan, cetostearyl alcohol, cetyl alcohol, cholesterol, diethanolamine, docusate salts, glyceryl monooleate, glyceryl monostearate, glyceryl palmitostearate, glyceryl monostearate, hectorite, hydroxypropyl cellulose, hydroxypropyl starch, lanolin, hydrous lanolin, lanolin alcohols, lauric acid, lecithin, linoleic acid, magnesium oxide, methylcellulose, Amerchol 1-101, monoethanolamine, myristic acid, myristyl alcohol, octyldodecanol, oleic acid, oleyl alcohol, pectin, phenylmercuric salts, phospholipids, poloxamer, polycarbophil, polyoxyethylene alkyl ethers, hydrogenated polyoxyl castor oil, polysorbate 20, polysorbate 40, polysorbate 60, polysorbate 90, polyoxyethylene stearates, polyoxylglycerides, caprylocaproyl macrogolglycerides, lauroyl macrogolglycerides, linoleoyl macrogolglycerides, oleoyl macrogolglycerides, stearoyl macrogolglycerides, potassium alginate, propylene glycol alginate, safflower glycerides, saponites, borax, sodium citrate, sodium lactate, sodium lauryl sulphate, sodium phosphate, sorbitan esters, sorbitan laurate, sorbitan oleate, sorbitan palmitate, sorbitan stearate, sorbitan trioleate, sorbitan sesquioleate, stearic acid, stearate salts, stearate esters, triethanolamine, vitamin E polyethylene glycol succinate, emulsifying wax, cetostearyl alcohol (type A), cetostearyl alcohol (type B), cetomacrogol emulsifying wax, xanthan gum, preferably cetomacrogol 1000 (ceteareth-20) and carbopol 940.

In some embodiments, the co-emulsifying agent(s) is/are used in an amount of 0.1%, 2%, 5%, 7%, 10%, 12%, 15%, or up to 35% by weight.

In some embodiments, compositions of the present invention comprise ingredients including, stabilizers including anionic, cationic or non-ionic surfactants, such as tween 20, tween 40, tween 60, tween 80, span 20, span 40, span 60, span 80, acacia, agar, bentonite, calcium alginate, carbomer, carboxymethylcellulose, carrageenan, cetostearyl alcohol, cetyl alcohol, cholesterol, diethanolamine, docusate salts, glyceryl monooleate, glyceryl monostearate, glyceryl palmitostearate, glyceryl monostearate, hectorite, hydroxypropyl cellulose, hydroxypropyl starch, lanolin, hydrous lanolin, lanolin alcohols, lauric acid, lecithin, linoleic acid, magnesium oxide, methylcellulose, Amerchol 1-101, monoethanolamine, myristic acid, myristyl alcohol, octyldodecanol, oleic acid, oleyl alcohol, pectin, phenylmercuric salts, phospholipids, poloxamer, polycarbophil, polyoxyethylene alkyl ethers, hydrogenated polyoxyl castor oil, polysorbate 20, polysorbate 40, polysorbate 60, polysorbate 90, polyoxyethylene stearates, polyoxylglycerides, caprylocaproyl macrogolglycerides, lauroyl macrogolglycerides, linoleoyl macrogolglycerides, oleoyl macrogolglycerides, stearoyl macrogolglycerides, potassium alginate, propylene glycol alginate, safflower glycerides, saponites, borax, sodium citrate, sodium lactate, sodium lauryl sulphate, sodium phosphate, sorbitan esters, sorbitan laurate, sorbitan oleate, sorbitan palmitate, sorbitan stearate, sorbitan trioleate, sorbitan sesquioleate, stearic acid, stearate salts, stearate esters, triethanolamine, vitamin E polyethylene glycol succinate, emulsifying wax, cetostearyl alcohol (type A), cetostearyl alcohol (type B), cetomacrogol emulsifying wax, xanthan gum, preferably cetomacrogol 1000 (ceteareth-20) and carbopol 940.

In some embodiments, the stabilizer(s) is/are used in an amount of 0.1%, 2%, 5%, 7%, 10%, 12%, 15%, or up to 35% by weight.

In some embodiments, compositions of the present invention comprise ingredients including, oil as a solubilizing agent for the cannabinoid component. This includes benzalkonium chloride, benzethonium chloride, benzyl benzoate, cetylpyridinium chloride, cyclodextrins, alfadex betadex, gamma cyclodextrin, glyceryl monostearate, hydroxypropylbetadex, hypromellose, lecithin, macrogol 15-hydroxystearate, meglumine, phospholipids, poloxamer, macrogol cetostearyl ether, macrogol lauryl ether, macrogol oleyl ether, macrogol stearyl ether, lauromacrogol, hydrogenated polyoxyl castor oil, polyoxyethylene sorbitan fatty acid esters, polyoxyl castor oil, polyoxyethylene sorbitan, fatty acid esters such as polyoxyethylene stearates, sulfobutylether β-cyclodextrin, glycerol trielaidate, or any suitable oil or waxy material, such as liquid paraffin, mineral oil, white mineral oil, any vegetable oil such as sunflower oil, Ω-3 fatty acids, benzyl alcohol, non-toxic organic solvents such as dimethyl sulfoxide, isopropyl alcohol or ethanol, glycerol, isopropyl myristate mixture or blend thereof.

In some embodiments, the solubilizing agent(s) is/are used in an amount of 0.1%, 2%, 5%, 7%, 10%, 12%, 15%, or up to 35% by weight.

In some embodiments, compositions of the present invention comprise ingredients including, emollient agents, such as almond oil, any tocopherol or mixture of tocopherols, aluminum monostearate, canola oil, castor oil, ceratonia, cetostearyl alcohol, cetyl alcohol, cholesterol, dimethicone, glycols such as glycerin or propylene glycol, glycerol dibehenate, glyceryl monooleate, glyceryl monostearate, glyceryl palmitostearate, glyceryl monostearate, isopropyl myristate, isopropyl palmitate, hydroxypropyl cellulose, hydroxypropyl starch, lanolin, hydrous lanolin, lanolin alcohols, lecithin, linoleic acid, mineral oil, Amerchol 1-101, methylcellulose, myristyl alcohol, octyldodecanol, oleic acid, oleyl alcohol, pectin, stearate esters, triethanolamine, vitamin E polyethylene glycol succinate, emulsifying wax, cetostearyl alcohol (type A), cetostearyl alcohol (type B), cetomacrogol emulsifying wax, xylitol, cetyl ester wax, zinc acetate, bees wax and mixtures or blends thereof.

In some embodiments, the emollient(s) is/are used in an amount of 1%, 2%, 5%, 7%, 10%, 12%, 15%, or up to 35% by weight.

In some embodiments, compositions of the present invention comprise ingredients including, viscosity modifying agents, such as acacia, agar, alginic acid, aluminum monostearate, aluminum magnesium silicate, cellulose derivatives such as carboxymethylcellulose calcium, carboxymethylcellulose sodium, carrageenan, ceratonia, cetostearyl alcohol, colloidal silicon dioxide, gelatin, glycerin, glyceryl behenate, hectorite, hydrophobic colloidal anhydrous silica, hydroxyethylcellulose, hydroxyethylmethylcellulose, hydroxypropylcellulose, hypromellose, hyaluronate derivatives such as sodium hyaluronate, methylcellulose, polydextrose, polyvinyl alcohol, povidone, saponite, sodium chloride, sucrose, β-cyclodextrin sulfobutylether, or its salts, tragacanth, paraffin, hard paraffin, bees wax, any suitable types of gum, pectin, hydrogenated vegetable oil type I, or any suitable resin and, preferably, myrrh resin or myrrh extract.

In some embodiments, the viscosity modifying agent(s) is/are used in an amount of 0.01%, 0.2%, 1%, 5%, 10%, 12%, 15%, or up to 35% by weight.

In some embodiments, compositions of the present invention comprise ingredients including, perfuming agents, such as, amyrin, camphor, cardinol, carvacrol, caryophyllene, citronellol, cycloartenol, cymene, fenchol, friedelin, isoborneol, isopulegol, elemene, eugenol, eucalyptol, eudesmol, geraniol, guaiol, germacrene, linalool, limonene, myrcene, menthol, ocimene, pinene, pulegone, phellandrene, sabinene, terpineol, terpinene, thujone, thymol, and the like or mixtures or blends thereof.

In some embodiments, the natural perfuming agent(s) is/are used in an amount of 0.0010%, 0.01%, 0.10%, 1%, 2%, 3% or up to 15% by weight.

In some embodiments, compositions of the present invention comprise ingredients including, preservative(s), such as benzalkonium chloride, benzethonium chloride, benzoic acid and its salt(s), benzyl alcohol, boric acid, bronopol, butylene glycol, parabens such as butylparaben or methylparaben, calcium acetate, calcium lactate, cetrimide, chlorhexidine base or salt(s), chlorobutanol, cresol derivatives such as chlorocresol, xylenol derivatives such as chloroxylenol, edetic acid, hexetidine, imidurea, monothioglycerol, phenoxyethanol, phenylethyl alcohol, phenylmercuric borate, phenylmercuric nitrate, potassium sorbate, sodium propyl hydroxybenzoate, sodium propionate, sodium lactate, sodium sulphite, sorbic acid, thiomersal, or mixtures, blends or combination thereof.

In some embodiments, the preservative(s) is/are used in an amount of 0.0001%, 0.001%, 0.01%, 0.1%, or up to 5% by weight.

In some embodiments, compositions of the present invention comprise ingredients including, natural preservative(s), such as thymol, carvacrol, eugenol, eucalyptol, geranyl acetate, citronellyl formate, cinnamaldehyde, citral, geranial, neral, ketones, carvone, menthone, citronellol, geraniol, linalool, menthol, limonene, pinene or mixtures, blends or combinations thereof.

In some embodiments, the natural preservative(s) is/are used in an amount of 0.001%, 0.01%, 0.10%, 1%, 2%, 3% or up to 5% by weight.

In some embodiments, compositions of the present invention can comprise one or more additional excipients or inactive materials, including coloring agents, opacifiers, pH controls, buffers, materials to control osmotic pressure, materials to help in the blending process, materials to help in any manufacturing or packaging process and the like.

In some embodiments, the present invention may be formulated as a facial serum, face cream, face lotion, antiaging facial serum, antiaging facial cream, antiaging facial lotion, antiaging facial mask, anti-wrinkle cream, anti-wrinkle serum, anti-wrinkle lotion, anti-wrinkle mask, glow oil, sleeping mask, eye serum, below eye cream, below eye mask, below eye lotion, body oil, recovery lotion, facial cream, lip cream, dual-phase topical preparation and the like.

Unless otherwise defined, all technical and scientific terms used herein have the same meaning as commonly understood by one of ordinary skill in the art to which this invention belongs. As used in the description of the invention and the appended claims, the singular forms “a,” “an,” and “the” are intended to include the plural forms as well, unless the context clearly indicates otherwise.

Unless otherwise indicated, all numbers expressing quantities of active or inactive ingredients, and so forth used in the specification and claims are to be understood as being modified in all instances by the term “about”. Accordingly, unless indicated to the contrary, the numerical parameters set forth in the specification included in this invention and the claims are approximations that may vary depending upon the desired properties sought to be obtained by the present invention. At the very least, each numerical parameter should be construed in light of the number of significant digits and ordinary rounding approaches.

Although the numerical ranges and parameters setting forth the scope of the invention are approximations, the numerical values set forth in the specific examples are reported to the best of the inventors' knowledge. Any numerical value; however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

In the present disclosure the term “about” can allow for a degree of variability in a value or range, for example, within 1%, within 5%, or within 10% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

As used herein, the terms “comprising”, “including”, “having” and grammatical variants thereof are to be taken as specifying the stated features, integers, steps, or components but do not preclude the addition of one or more additional features, integers, steps, components or groups thereof.

As used herein, the following terms and phrases shall have the meanings set forth below. Unless defined otherwise, all technical and scientific terms used herein have the same meaning as commonly understood to one of ordinary skill in the art.

The terms “pharmaceutically acceptable diluent” and “pharmaceutically acceptable excipient” are art-recognized and refer to a pharmaceutically acceptable material, composition or vehicle, such as a liquid or solid filler, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose and maltose; (2) starches, such as corn starch and gelatinized starch; (3) cellulose, and its derivatives, such as carboxymethyl cellulose salt, and hydroxypropylmethyl cellulose; (4) thickening agents such as gelatin and tragacanth; (5) disintegrants such as copovidone; (6) other excipients, such as cocoa butter and suppository waxes and pyrogen-free water for sterile products; and (7) other non-toxic compatible substances employed in pharmaceutical formulations.

The term “subject” or “patient” in the present disclosure refers to human patients but is not limited to humans and may include animals.

As used herein, the term “administering” includes all means of introducing the compositions described herein to the subject, including mainly topical administering to any part of the subject. The compositions described herein may be administered in unit dosage forms or formulations containing conventional nontoxic pharmaceutically acceptable carriers, adjuvants, and vehicles.

Unless indicated otherwise, percent is weight percent and ratio is weight/weight ratio. Unless indicated otherwise, weight ratio means the ratio between weight content, e.g., in an aqueous solution containing 10% solute and 90% water, the solute to water weight ratio is 1:9.

Unless indicated otherwise, the term “cannabinoid” as used herein refers to a compound that affects the endocannabinoid system. Cannabinoids are agonists or antagonists to receptors in the endocannabinoid system.

As used herein, the term “herbal” refers to natural products or extracts isolated from plants, animal, algae and bacteria and the like.

As used herein, the term “species” refers to specific plant examples but may include other related plants in the same genus and family known to those skilled in this field to produce the same effects.

As used herein, the term “extract” refers to compounds from plants that have been extracted and concentrated using one of the many known extraction methods, including hydrocarbon and non-hydrocarbon solvent extracts, maceration, percolation, decoction, reflux extraction, Soxhlet extraction, pressure liquid extraction, supercritical fluid extraction, ultrasonic assisted extraction, microwave assisted extraction, pulsed electrical field extraction, enzyme assisted extraction, hydro distillation and steam distillation, affinity extraction or other extraction methods, or combination of methods known to those skilled in this field.

As used herein, the term “cellulose” refers to cellulose, hemicellulose, and their combinations.

As used herein, the term “glycol” refers to any glycol, including ethylene glycol, polyethylene glycol, propylene glycol and polypropylene glycol.

### EXAMPLES

### Example 1: Topical Moisturizing and Antiaging Cream Formula, Wherein Each 100 g of Cream Contains the Following Formula

| | Quantity | |
|---|---|---|
| Ingredient | (mg) | Function |
| Cannabidiol | 500 | Active component |
| Opuntia fruit extract | 2500 | Active component |
| Hyaluronic acid | 0.1 | Active component |
| Sorbitan monostearate | 0.75 | Emulsifier |
| Tween 60 | 3 | Emulsifier |
| Cetostearyl alcohol | 6 | Stabilizer |
| Propylene glycol | 5 | Solvent and emollient |
| Benzyl alcohol | 1 | Co- solvent |
| Methyl paraben | 0.14 | Preservative |
| BHT (Butylated hydroxytoluene) | 0.02 | Antioxidant |
| Medium chain triglycerides | 4 | Cream Base |
| Eicosapentaenoic acid | 2 | Cream Base |
| α-Linolenic acid | 2 | Cream Base |
| Isopropyl myristate | 5 | Emollient & skin |
| | | penetration enhancer |
| Purified water | to 100 g | Vehicle |

### Example 2: Face Moisturizing and Antiaging Lotion Formula, Wherein Each 100 g of Lotion Contains the Following Formula

| | Quantity | |
|---|---|---|
| Ingredient | (mg) | Function |
| Cannabidiol | 500 | Active component |
| Opuntia fruit extract | 2500 | Active component |
| Papain | 500 | Active component |
| Hyaluronate sodium | 0.2 | Active component |
| Sorbitan monostearate | 0.75 | Emulsifier |
| Tween 80 | 3 | Surfactant |
| Cetostearyl alcohol | 6 | Stabilizer |
| Propylene glycol | 5 | solvent |
| Benzyl alcohol | 1 | Co- solvent |
| Methyl paraben | 0.14 | Preservative |
| Thymol | 1 | Antioxidant |
| Medium chain triglycerides | 2 | Cream Base |
| Isopropyl myristate | 5 | Emollient & skin |
| | | penetration enhancer |
| Purified water | to 100 g | Vehicle |

### Example 3: Topical Herbal Under-Eye Antiwrinkle Cream, Wherein Each 100 g of Cream Contains the Following Formula

| | Quantity | |
|---|---|---|
| Ingredient | (mg) | Function |
| Cannabidiol | 1000 | Active component |
| Opuntia fruit extract | 5000 | Active component |
| Papaya extract | 250 | Active component |
| Hyaluronate sodium | 0.2 | Active component |
| Tween 60 | 5 | Emulsifier |
| Cetomacrogol Emulsifying Wax | 5 | Stabilizer |
| Citronellol | 0.1 | Perfuming agent |
| Propylene glycol | 5 | Solvent and emollient |
| Myrrh resin | 1 | Viscosity modifier |
| Thymol | 0.15 | Natural Preservative |
| Cinnamaldehyde | 0.15 | Natural Preservative |
| Ascorbyl palmitate | 0.5 | Antioxidant |
| Fish oil | 4 | Cream Base |
| Eicosapentaenoic acid | 2 | Cream Base |
| α-Linolenic acid | 2 | Cream Base |
| Isopropyl myristate | 5 | Emollient & skin |
| | | penetration enhancer |
| Purified water | to 100 g | Vehicle |

The present invention has been described and illustrated with reference to an exemplary embodiment; however, it will be understood by those skilled in the art that various changes may be made, and equivalents may be substituted for elements thereof without departing from the scope of the invention as set out in the following claims. Therefore, it is intended that the invention is not limited to the embodiments disclosed herein.

What is claimed is:

1. A skin care formulation, comprising: a cannabinoid, an herbal extract, and at least 1×10⁻⁴% by weight of a hyaluronate derivative.

2. The skin care formulation of claim 1, comprising: at least 5×10⁻³% by weight of a pharmaceutically acceptable skin penetration enhancer, at least 2×10⁻³% by weight of a pharmaceutically acceptable oleaginous base, and at least 5×10⁻³% by weight of a pharmaceutically acceptable emollient.

3. The skin care formulation of claim 2, wherein the herbal extract is an extract of an edible Opuntia species.

4. The skin care formulation of claim 3, comprising an extract from a papaya species.

5. The skin care formulation of claim 4, wherein the extract from a papaya species comprises one or more proteolytic enzymes.

6. The skin care formulation of claim 5, wherein the one or more proteolytic enzymes are one or both papain and bromelain.

7. The skin care formulation of claim 3, comprising one or more additional herbal extracts selected from the group consisting of: myrrh, myrrh resin, myrrh volatile oil, myrrhin, Salvadora persica, Nigella sativa, Glycyrrhiza glabra, Camellia sinensis, Aloe vera, terpene (e.g. β-caryophyllene), collagen, Albizia julibrissin, squalane, Prunus amygdalus dulcis, Tephrosia purpurea, probiotic, Adansonia digitata, yeast, Extrait de Levure, Cola acuminata, Anthemis nobilis, Dipsacus sylvestris, Silybum marianum, Chenopodium quinoa seed, Avena sativa (oat) kernel, Theobroma cacao, Leontopodium alpinum, Musa sapientum, Salicornia herbacea, Actinidia chinensis, Kalanchoe pinnata, Myrothamnus flabellifolia, Lycium barbarum, Orthosiphon stamineus, Hedychium coronarium, Mangifera indica, Jania rubens, Engelhardtia chrysolepis, Callicarpa japonica, retinoids, argan, tiger grass, shea butter, adapalene, hydroquinone, Limnanthes alba (meadowfoam), coconut, Withania somnifera (ashwagandha), Helianthus annuus (sunflower), Helianthus annuus, Lentinus edodes (shiitake), Borago officinalis, tocopheryl acetate (vitamin E), Helichrysum stoechas, Melia azadirachta, Melia azadirachta, amino esters, Mimosa tenuiflora, Coccinia indica, Solanum melongena (eggplant), Aloe barbadensis, Ocimum sanctum, Ocimum basilicum, Curcuma longa (turmeric), Corallina ocinalis, marula, argan, lavender oil, sunflower, Simmondsia chinensis (jojoba), Prunus armeniaca (apricot) kernel, Borago officinalis (borage), Punica granatum (pomegranate), Cucumis sativus (cucumber), Vaccinium macrocarpon (cranberry), Brassica oleracea italica (broccoli), Persea gratissima (avocado), Coffea arabica (green coffee), Calendula officinalis (Calendula), Centella asiatica (gotu kola), Equisetum arvense (horsetail), Urtica dioica (nettle), Hippophae rhamnoides (sea buckthorn) fruit, Rosmarinus officinalis (rosemary), Citrus aurantium (bergamot), Citrus limon (lemon), Pelargonium graveolens (geranium), Boswellia carteri (frankincense), Jasminum grandiflorum (jasmine), Citrus aurantium (neroli), Daucus carota (carrot seed), Calendula, sandalwood, and tea rose extracts.

8. The skin care formulation of claim 3, comprising one or more pharmaceutically acceptable compounds selected from the group consisting of phytochemicals, exfoliating agents, UV blocking agents, skin penetration enhancers, oleaginous bases, antioxidants, emulsifying agents, co-emulsifying agents, stabilizers, solubilizing agents, emollients, viscosity modifying agents, natural preservatives, synthetic preservatives, natural perfuming agents, flavoring agents, and coloring agents.

9. The skin care formulation of claim 8, wherein each of the one or more pharmaceutically acceptable compounds, if present in the skin care formulation, are present in the following amounts: between 5×10⁻³% and 15% by weight of skin penetration enhancer, between 2×10⁻³ and 25% by weight of oleaginous base, between 5×10⁻³ and 15% by weight of emollient, between 2×10⁻⁵ and 5% by weight of antioxidant, between 7.5×10⁻⁴ and 15% by weight of emulsifying agent, between 1×10⁻³ and 15% by weight of co-emulsifying agent, between 5×10⁻³ and 15% by weight of stabilizer, between 5×10⁻³ and 15% by weight of solubilizing agent, and between 1×10⁻³ and 15% by weight of viscosity modifying agent.

10. The skin care formulation of claim 9, wherein each of the one or more pharmaceutically acceptable compounds, if present in the skin care formulation, are present in the following amounts: between 5×10⁻³% and 7% by weight of skin penetration enhancer, between 2×10⁻³ and 10% by weight of oleaginous base, between 5×10⁻³ and 7% by weight of emollient, between 2×10⁻⁵ and 1% by weight of antioxidant, between 7.5×10⁻⁴ and 7% by weight of emulsifying agent, between 1×10⁻³ and 7% by weight of co-emulsifying agent, between 5×10⁻³ and 7% by weight of stabilizer, between 5×10⁻³ and 7% by weight of solubilizing agent, and between 1×10⁻³ and 5% by weight of viscosity modifying agent.

11. The skin care formulation of claim 9, wherein the exfoliating agent is selected from the group consisting of: salicylic acid, glycolic acid, malic acid, citric acid, and salts, hydrates, and combinations thereof.

12. The skin care formulation of claim 9, wherein the UV blocking agent is selected from the group consisting of curcumin derivatives, coconut oil, titanium dioxide, zinc oxide, butyl methoxy dibenzoylmethane, para-aminobenzoic acid and its derivatives, cinnamates, octyl methoxy cinnamate, salicylates, camphor derivatives, avobenzone, triazones, benzophenones, and combinations thereof.

13. The skin care formulation of claim 9, wherein the skin penetration enhancer is selected from the group consisting of dimethyl sulfoxide (DMSO), lanolin, wool fat, polyethylene glycol, caprylic acid triglyceride (Tricaprylin), and myristate esters.

14. The skin care formulation of claim 9, wherein the oleaginous base is selected from the group consisting of liquid paraffin, mineral oil, white mineral oil, vegetable oil, Ω-3 fatty acids, eicosapentaenoic acid, α-linolenic acid, fish oil, triglycerides, medium chain triglycerides, cetostearyl ether, macrogol lauryl ether, macrogol oleyl ether, macrogol stearyl ether, lauromacrogol, hydrogenated polyoxyl castor oil, fatty acid esters, ethyl stearate, and mixtures and blends thereof.

15. The skin care formulation of claim 9, wherein the antioxidant is selected from the group consisting of: vitamin A, vitamin E, vitamin C, ascorbyl palmitate, ascorbate salts, stilbene antioxidant derivatives, hydroxytoluene derivatives, butylated hydroxytoluene (BHT), hydroxyanisole, butylated hydroxyanisole (BHA), pineapple extract, citric acid monohydrate, edetic acid, erythorbic acid, fumaric acid, malic acid, methionine, monothioglycerol, pentetic acid, phosphoric acid, metabisulphite salts, propionic acid, gallate esters, sodium formaldehyde sulfoxylate, sodium pyrosulfite, sulfite salts, thiosulphate salts, sulfur dioxide, tartaric acid, thymol, vitamin E polyethylene glycol succinate, glycyrrhizic acid, and mixtures and blends thereof.

16. The skin care formulation of claim 15, wherein the antioxidant is one or more stilbene antioxidant derivatives.

17. The skin care formulation of claim 16, wherein the one or more stilbene antioxidant derivatives are one or both piceatannol and resveratrol.

18. The skin care formulation of claim 9, wherein the solubilizing agent is selected from the group consisting of: benzalkonium chloride, benzethonium chloride, benzyl benzoate, cetylpyridinium chloride, cyclodextrins, alfadex betadex, gamma cyclodextrin, glyceryl monostearate, hydroxypropylbetadex, hypromellose, lecithin, macrogol 15-hydroxystearate, meglumine, phospholipids, poloxamer, macrogol cetostearyl ether, macrogol lauryl ether, macrogol oleyl ether, macrogol stearyl ether, lauromacrogol, hydrogenated polyoxyl castor oil, polyoxyethylene sorbitan fatty acid esters, polyoxyl castor oil, polyoxyethylene sorbitan, polyoxyethylene stearates, sulfobutylether β-cyclodextrin, glycerol trielaidate, liquid paraffin, mineral oil, white mineral oil, vegetable oil, sunflower oil, Ω-3 fatty acids, benzyl alcohol, dimethyl sulfoxide, isopropyl alcohol, ethanol, glycerol, isopropyl myristate, and mixtures and blends thereof.

19. The skin care formulation of claim 9, wherein the cannabinoid component is one or more cannabinoids selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof.

20. The skin care formulation of claim 19, wherein the cannabinoid is CBD.

21. The skin care formulation of claim 19, wherein the cannabinoid is hemp oil.

22. The skin care formulation of claim 9, wherein each 100 g of the formulation comprises 500 mg of cannabidiol, 2500 mg of Opuntia fruit extract, 0.1 mg of hyaluronic acid, 0.75 mg of sorbitan monostearate, 3 mg of tween 60, 6 mg of cetostearyl alcohol, 5 mg of propylene glycol, 1 mg of benzyl alcohol, 0.14 mg of methyl paraben, 0.02 mg of butylated hydroxytoluene, 4 mg of medium chain triglycerides, 2 mg of eicosapentaenoic acid, 2 mg of α-Linolenic acid, 5 mg of isopropyl myristate, and purified water to fill to 100 g.

23. The skin care formulation of claim 9, wherein each 100 g of the formulation comprises 500 mg of cannabidiol, 2500 mg of Opuntia fruit extract, 500 mg of papain, 0.2 mg of hyaluronate sodium, 0.75 mg of sorbitan monostearate, 3 mg of tween 80, 6 mg of cetostearyl alcohol, 5 mg of propylene glycol, 1 mg of benzyl alcohol, 0.14 mg of methyl paraben, 1 mg of thymol, 2 mg of medium chain triglycerides, 5 mg of isopropyl myristate, and purified water to fill to 100 g.

24. The skin care formulation of claim 9, wherein each 100 g of the formulation comprises 1000 mg of cannabidiol, 5000 mg of Opuntia fruit extract, 250 mg of papaya extract, 0.2 mg of hyaluronate sodium, 5 mg of tween 60, 5 mg of cetomacrogol emulsifying wax, 0.1 mg of citronellol, 5 mg of propylene glycol, 1 mg of myrrh resin, 0.15 mg of thymol, 0.15 mg of cinnamaldehyde, 0.5 mg of ascorbyl palmitate, 4 mg of fish oil, 2 mg of eicosapentaenoic acid, 2 mg of α-Linolenic acid, 5 mg of isopropyl myristate, and purified water to fill to 100 g.

25. A method of treating, alleviating, or delaying dermatological conditions or symptoms by administering to a subject in need a therapeutically effective amount of a skin care formulation according to any of claims 1 to 24.
